(19) United States
(12) Patent Application Publication
(10) Pub. No.: US 2006/0234251 A1
(43) Pub. Date: Oct. 19, 2006

(54) Methods of enhancing isolation of RNA from biological samples

(75) Inventor: Hashem Akhavan-Tafti, Howell, MI (US)

Correspondence Address: LUMIGEN, INC., 22900 W. EIGHT MILE ROAD, SOUTHFIELD, MI, 48034, US

(73) Assignee: Lumigen, Inc.

(21) Appl. No.: 11/109,366

(22) Filed: Apr. 19, 2005

(51) Int. Cl. C12Q 1/68; C07H 21/04

(52) U.S. Cl. 435/6; 536/25.4

(57) ABSTRACT

A method of enhancing the isolation of ribonucleic acids from samples of biological or cellular material is disclosed which uses a solution of an Agent during binding of nucleic acids onto a solid phase binding material which, after elution, enhances the recovery of RNA. Washing the solid phase and eluting the nucleic acid produces RNA in enhanced yield and/or purity. The use of the new method allows RNA to be captured and released in a form suitable for downstream processing in under five minutes. Preferred Agents according to the invention comprise monomeric, oligomeric, dendrimeric and polymeric organic compounds having multiple positive charges.

### FIELD OF THE INVENTION

The present invention relates generally to the isolation of ribonucleic acids from biological samples, and more specifically to a method of isolation employing novel solutions for enhancing recovery of RNA.

### BACKGROUND OF THE INVENTION

Modern molecular biology methods as applied to clinical research, clinical diagnostic testing, and drug discovery have made increasing use of the study of ribonucleic acid (RNA). RNA is present as messenger RNA (mRNA), transfer RNA (tRNA) and ribosomal RNA (rRNA). Studies of the presence of particular mRNA sequences and levels of expression of mRNAs have become prevalent. Analysis of mRNA, especially using microarrays, is a very powerful tool in molecular biology research. By measuring the levels of mRNA sequences in a sample, the up- or down-regulation of individual genes is determined. Levels of mRNA can be assessed as a function of external stimuli or disease state. For example, changes in p53 mRNA levels have been positively associated with cancer in multiple cell types. Additionally, a number of viruses with a significant impact on human health, including HIV, are RNA viruses. The ability to rapidly and cleanly isolate viral RNA from bodily fluids or tissues is important in virology research.

Current methods for isolating RNA begin with one of a variety of techniques to disrupt cells, liberate RNA into solution, and protect RNA from RNases. Lysis liberates RNA along with DNA and protein from which the RNA must then be separated. Thereafter, the RNA is treated either to solubilize it or to precipitate it. The use of chaotropic guanidinium salts to simultaneously lyse cells, solubilize RNA and inhibit RNases was disclosed in Chirgwin et al, Biochem., 18, 5294-5299 (1979). Other methods free solubilized RNA from protein and DNA by extraction with phenol/chloroform at low pH (D. M. Wallace, Meth. Enzym., 15, 33-41 (1987)). A commonly used single step isolation of RNA involves treating cells sequentially with 4 M guanidinium salt, sodium acetate (pH 4), phenol, and chloroform/isoamyl alcohol. Samples are centrifuged and RNA is precipitated from the upper layer by the addition of alcohol (P. Chomczynski, Anal. Biochem., 162, 156-159 (1987)). U.S. Pat. No. 4,843,155 describes a method in which a stable mixture of phenol and guanidinium salt at an acidic pH is added to the cells. After phase separation with chloroform, the RNA in the aqueous phase is recovered by precipitation with an alcohol.

Other methods include adding hot phenol to a cell suspension, followed by alcohol precipitation (T. Maniatis et al, Molecular Cloning, A Laboratory Manual, Cold Spring Harbor Laboratory (1982)); the use of anionic or non-ionic surfactants to lyse cells and liberate cytoplasmic RNA; and the use of inhibitors of RNases such as vanadyl riboside complexes and diethylpyrocarbonate [L. G. Davis et al, “Guanidine Isothiocyanate Preparation of Total RNA” and “RNA Preparation: Mini Method” in Basic Methods in Molecular Biology, Elsevier, N.Y., pp. 130-138 (1991).

A technique for isolating both DNA and RNA from biological sources by binding on glass or other solid phases was disclosed in U.S. Pat. No. 5,234,809 (Boom et al.). Cells contained within biological sources, such as serum or urine, were lysed by exposure to strong (>5 M) solutions of guanidinium thiocyanate in Tris HCl (pH 8.0), containing EDTA and the surfactant Triton X-100. DNA and RNA were purified from biological materials by incubation with diatomaceous earth or silica particles, which formed reversible complexes with the DNA and RNA.

U.S. Pat. No. 5,155,018 provides a process for isolating and purifying biologically active RNA from a biological source, which may also include DNA, proteins, carbohydrates and other cellular materials. RNA is isolated by contacting the biological source with finely divided glass or diatomaceous earth in the presence of a binding solution comprising concentrated, acidified chaotropic salt. Under these conditions, it is claimed that RNA binds selectively to the particulate siliceous material although subsequent treatment of the solid material with ethanolic salt solution to remove DNA is also disclosed. The particle-bound RNA can be easily separated from the other biological substances contained in the sample. Preferably, the particle-bound RNA is washed to remove non-specifically adsorbed materials. The bound RNA is released from the particles by elution with a dilute salt buffer, and the substantially pure, biologically active RNA is recovered. Addition of a nuclease to destroy DNA in the eluant is also disclosed, calling into further question the claim of selective binding of RNA.

The ability of singly charged monomeric cationic surfactants to lyse cells and simultaneously precipitate RNA and DNA from solution was described in U.S. Pat. Nos. 5,010,183 and 5,985,572. In these patents RNA is first rendered insoluble. In the method of the '183 patent, a solution of the quaternary ammonium surfactant together with 40% urea and other additives is added to a cell suspension, and the mixture is centrifuged. The pellet is resuspended in ethanol, from which nucleic acids are precipitated by addition of a salt.

The cumbersome multi-step nature of the above methods for isolating RNA complicates the use of RNA in clinical practice. Methods must overcome the difficulty of separating RNA from the protein and DNA in the cell before the RNA is degraded by nucleases, such as RNase. These nucleases are present in blood in sufficient quantities to destroy unprotected RNA rapidly. Successful methods for the isolation of RNA from cells must therefore be capable of preventing degradation by RNases. There remains a need in the art for a rapid, simple method for isolating RNA from biological samples which method minimizes hydrolysis and degradation of the RNA so that it can be used in various analyses and downstream processes.

### SUMMARY OF THE INVENTION

In one aspect, the present invention provides a novel method for enhanced isolation of RNA from a biological sample, including cell culture, involving the use of an Agent for enhancing recovery of RNA, hereinafter termed ‘Agent’ and a solid phase binding material. The Agent can comprise a multiply charged compound. The Agent can further comprise a quaternary onium compound.

In another aspect, the invention provides a kit for isolating and purifying RNA from a biological sample which contains at least an Agent and a solid phase binding material as described herein.

### BRIEF DESCRIPTION OF THE DRAWINGS

FIG. 1 is a photograph of a gel showing the isolation of RNA from varying volumes of E. Coli/pUC18 culture. Culture aliquots from 50 μL to 5 mL were pelleted, resuspended in 1 mL of 0.5% Compound 1, bound onto magnetic particles, washed with 10 mM Tris, pH 8.0, and eluted with 0.1 M NaOH solution. The gel results shown in FIG. 1 reveal that RNA was isolated selectively in as little as 250 μL of culture under these conditions. Treating an aliquot of each eluent with RNase A (0.02%) confirmed the identity of the band as RNA.

### DETAILED DESCRIPTION OF THE INVENTION

Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Biological material—includes whole blood, anticoagulated whole blood, tissue, cells, cellular content, extracellular nucleic acids, and viruses.

Cellular material—intact cells or material, including tissue, containing intact cells of animal, plant or bacterial origin.

Cellular nucleic acid content—refers to nucleic acid found within cellular material and can be genomic DNA and RNA, and other nucleic acids such as that from infectious materials, including viruses and plasmids.

Magnetic particle—a particle, microparticle or bead that is responsive to an external magnetic field. The particle may itself be magnetic, paramagnetic or superparamagnetic. It may be attracted to an external magnet or applied magnetic field as when using superparamagnetic or ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Oligomer, oligonucleotide—as used herein will refer to a compound containing a phosphodiester internucleotide linkage and a 5′-terminal monophosphate group. The nucleotides can be the normally occurring ribonucleotides A, C, G, and U or deoxyribonucleotides, dA, dC, dG and dT.

Nucleic acid—A polynucleotide can be DNA, RNA or a synthetic DNA analog such as a PNA. Single stranded compounds and double-stranded hybrids of any of these three types of chains are also within the scope of the term

Release, elute—to remove a substantial portion of a material bound to the surface or pores of a solid phase material by contact with a solution or composition.

RNA—includes messenger RNA (mRNA), transfer RNA (tRNA) and ribosomal RNA (rRNA).

Sample—A fluid containing or suspected of containing nucleic acids. Typical samples which can be used in the methods of the invention include bodily fluids such as blood, which can be anticoagulated blood as is commonly found in collected blood specimens, plasma, serum, urine, semen, saliva, cell cultures, tissue extracts and the like. Other types of samples include solvents, seawater, industrial water samples, food samples and environmental samples such as soil or water, plant materials, cells originated from prokaryotes, eukaryotes, bacteria, plasmids and viruses.

Solid phase material—a material having a surface to which can attract nucleic acid molecules. Materials can be in the form of particles, microparticles, nanoparticles, fibers, beads, membranes, filters and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

1. The RNA Isolation Method of the Invention

The present invention provides methods for enhancing the isolation of RNA from biological samples using an Agent as defined above and a solid phase binding material which method demonstrates significant advantages over other known methods. Applicant's co-pending U.S. patent application Ser. No. 11/061,984, filed on Mar. 2, 2005 discloses solid phase binding materials for rapidly isolating nucleic acids from samples of biological materials. Applicant has surprisingly discovered that addition of certain compounds (Agents) to a sample prior to or concurrently with binding the nucleic acid to the solid phase enhances the isolation of RNA by increasing the quantity of RNA produced and/or allowing RNA to be selectively isolated from genomic DNA and other forms of DNA such as plasmid DNA. The method of the present invention relies on the use of these Agents which, as demonstrated in the examples below, are unexpectedly effective in causing RNA to be released from e.g. viruses, cells and tissues containing intact cells.

RNA can be isolated and purified according to the process of the invention from any biological fluid, e.g. virus, cell lysate or tissue homogenate. Common sources include, but are not limited to, bacterial culture or pellets, blood, urine, cells, and lysates of tissues or cells. The method of the invention can be applied to samples including lysates or intact cells and tissues, such as cultured bacterial, plant or animal cell lines and tissues whether or not they have been subject to other preliminary procedures. In particular, nucleic acids produced through preliminary ultrasonic disruption, or lysis and ethanol precipitation, or through lysis with moderate to high concentration guanidinium salt solutions can be used as the starting point in the present method. Alternatively, no preliminary disruption or lysis need be used at all. Purification of RNA from cells in suspension, i.e., from biological fluids or cell culture, can begin, for example, by pelleting cells with low-speed centrifugation and discarding the medium. RNA may be purified from intact tissues or organs by homogenizing, using a hand held homogenizer or an automatic homogenizer, such as a Waring blender, or other tissue homogenizer. The homogenate may be passed through a coarse filter, such as cheesecloth, to remove large particulate matter or the preparation may be centrifuged at low speed to separate particulate material.

In the method of the invention, a biological sample, prepared if necessary as described above, is mixed with a solution of an Agent of the invention. Contact of the sample with the Agent according to this method may promote lysis of cells in the sample and other interactions with nucleic acids. The RNA is, either concurrently or subsequently, adsorbed or otherwise bound to a solid phase material. The RNA is further isolated by washing the solid phase containing RNA to remove contaminants. Finally, the RNA is released from the solid phase by elution with an elution reagent. All of the steps can be performed rapidly, in succession, in a single container or on a single support without the need for specialized equipment such as centrifuges. The method is adaptable to automated platforms for processing large numbers of samples in serial or parallel fashion.

The method of this invention provides a convenient method for extracting RNA from biological samples. The method of this invention is rapid, typically requiring only a few minutes to complete. Significantly, the RNA obtained by the method is of an adequate purity such that it is useful for clinical or other downstream uses, such as the use of reverse transcriptase followed by the polymerase chain reaction (RT-PCR), cloning, amplification, RNA blot analysis and in vitro translation. Advantageously, it is not necessary to isolate cells prior to use of this method and only simple equipment is required for performance of the method. No preliminary lysis and ethanol precipitation step is necessary before processing samples in accordance with the method of the invention.

In the practice of the present invention, a selected biological sample, e.g., a pelleted portion of a cell culture, is mixed rapidly with a solution of an Agent described herein. Generally when a pelleted sample of bacterial culture is used as the sample, the pellet from 1 to 5 mL of culture is resuspended in 1 mL of Agent solution. Other quantities of culture, pellet and Agent solution can be used as determined by routine experimentation. The pellet and Agent need only be in contact in the mixture for between about a few seconds up to about 5 minutes. No other processing is needed.

After the formation of the mixture for the selected time, it is added to a solid phase. The supernatant is removed and the solid phase containing the nucleic acid is optionally washed with a wash solution. The solid phase is then eluted to solubilize the RNA and dissociate it from the solid phase. In one embodiment, an alkaline solution is used to elute the RNA from the surfactant/RNA complex. Typically, a desirable concentration of alkali for this purpose is from about 1 mM to about 1 M.

The quality and quantity of the extracted RNA is also enhanced by the optional addition of an RNase inhibitor, such as aurin tricarboxylic acid, DTT, or DEPC, to the extracting solvent. Other inhibitors of RNase may be selected for this purpose by the skilled person.

While it is not necessary to perform any preliminary cell disruption or lysis on samples before performing the methods of the invention, the methods can be applied to solutions of nucleic acids so treated. That is, two step processes employing pretreatments are specifically included within the scope of the presently described methods. Such pretreatments include the use of substances which promote cellular lysis or stabilization of RNA or inactivation of RNase enzymes, e.g. chaotropes such as guanidinium salts.

2. The Agent of the Invention

Substances useful as ADDITIVE in accordance with the invention comprise those materials or compounds which, when used to reconstitute a sample of biological material, permit the enhanced or selective isolation of RNA from the sample of biological material when treated with a solid phase nucleic acid binding material, in comparison to the isolation performed using water or a buffered medium for reconstituting the sample. Substances functioning as an Agent of the invention are preferably selected from multiply charged compounds containing at least two charged groups. More preferred are those compounds where the charges are permanent and not the result of pH changes. Examples of the Agent include dicationic compounds described in U.S. Pat. No. 5,451,347 comprising phosphonium and ammonium salt dicationic surfactants useful in practicing the present invention may be of the formula:
X⁻⁺R₃ACH₂-Link-CH₂AR₃⁺X⁻
wherein A may be P or N atoms and wherein Link is an organic spacer group containing two or more carbon atoms selected from the group consisting of substituted and unsubstituted aryl, alkyl, alkenyl, alkynyl and wherein Link may contain heteroatoms and wherein R is selected from lower alkyl or aralkyl containing 1 to 20 carbon atoms and wherein X is an anion. The R groups on a specific phosphorus or nitrogen atom may all be the same group or may be two different groups or all three may be different. The set of R groups on neighboring phosphorus or nitrogen atoms in the same molecule may be the same set or may be different sets wherein the sets are subject to the description above. An exemplary group of dicationic compounds is of the formula: 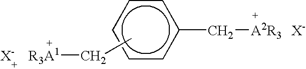
wherein the substituents may be in the ortho-, meta- or para-orientation and wherein A¹ and A¹ are independently chosen from nitrogen or phosphorus atoms, and wherein each R is independently alkyl or aralkyl containing from 1-20 carbon atoms and wherein X is an anion. Representative compounds in this group include compounds designated 1-23 in commonly assigned U.S. Pat. No. 5,439,617.

Another exemplary group of dicationic compounds is of the formula:
X⁻⁺R₃A¹CH₂-Link-CH₂A²R₃⁺X⁻
wherein A¹ and A¹ are independently chosen from nitrogen or phosphorus atoms and wherein Link is an organic spacer group containing two or more carbon atoms selected from the group consisting of substituted and unsubstituted alkyl, alkenyl, alkynyl, and wherein each R is independently alkyl or aralkyl containing from 1-20 carbon atoms and wherein X is an anion. Representative compounds in this group include compounds designated 24-27 wherein Link is an alkylene group in commonly assigned U.S. Pat. No. 5,439,617.

Another exemplary group of Agents comprise polymers having charged groups as part of the polymer backbone or as pendant groups. An exemplary group of polymers has the formula: 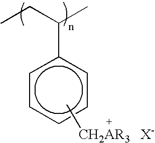
wherein each A is selected from phosphorus and nitrogen atoms, each R is selected from lower alkyl or aralkyl containing 1 to 20 carbon atoms, and wherein n is an integer between about 10 and 1000. The R groups on a specific phosphorus or nitrogen atom may all be the same group or may be two different groups or all three may be different. The set of R groups on adjacent phosphorus or nitrogen atoms may be the same set or may be different sets wherein the sets are subject to the description above. The relative position of substituents on the aromatic ring may be ortho, meta, para or mixtures of the three types in any proportion. Exemplary polymeric compounds are described in commonly assigned U.S. Pat. No. 5,393,469.

Another exemplary group of Agents comprise linear, branched oligomeric or dendrimeric polyionic compounds.

The anion in the above exemplary monomeric and polymeric Agents include halides, especially chloride, bromide and iodide, hydroxide, nitrate, sulfate, phosphate, alkyl and arylcarboxylate such as formate, acetate, propionate, benzoate, dicarboxylates including oxalate, malonate and succinate and other polycarboxylates such as citrate.

For use in the method of the present invention in isolating RNA, the Agents are in an aqueous solution at a concentration effective to promote the isolation of RNA in the present methods. Useful concentrations will generally fall within a range varying from high to low of a factor of ten or more. The bounds of this range can be readily determined empirically by application of the methods described in detail below in the specific examples. Specific useful conditions are identified in the examples as well. The aqueous solution of Agent can optionally contain an RNase inhibitor substance in an amount effective to inhibit decomposition of RNA. Diethylpyrocarbonate (DEPC) is a common RNase inhibitor. Mercaptan compounds including 2-mercaptoethanol and dithiothreitol are also known RNase inhibitors.

3. Solid Phase Materials

The RNA isolation methods of the present invention, which show enhanced recovery of RNA due to treating the sample with an Agent utilizes a solid support to bind the RNA, thereby separating the RNA from other sample components. Any solid matrix capable of binding RNA from a sample treated with an Agent according to the invention may be used. The materials for binding nucleic acids in the methods of the present invention comprise a matrix which defines its size, shape, porosity, and mechanical properties, and can be in the form of particles, microparticles, fibers, beads, membranes, and other supports such as test tubes and microwells. Preferred solid phase materials include silica, glass, sintered glass, controlled pore glass, sintered glass, alumina, zirconia, titania, insoluble synthetic polymers, insoluble polysaccharides, and metallic materials selected from metals, metal oxides, and metal sulfides, as well as magnetically responsive materials coated with silica, glass, synthetic polymers, or insoluble polysaccharides. Exemplary materials include silica based materials coated or functionalized with covalently attached surface functional groups that serve to disrupt cells and attract nucleic acids. Also included are suitably surface-functionalized carbohydrate based materials, and polymeric materials having this surface functionality. Numerous specific materials and their preparation are described in Applicant's co-pending U.S. application Ser. Nos. 10/714,763, 10/715,284, 10/891,880, 10/942,491, 60/638,631, and 11/061,984.

In one embodiment the materials further comprise a covalently linked nucleic acid binding portion at or near the surface which permits capture and binding of nucleic acid molecules of varying lengths. By surface is meant not only the external periphery of the solid phase material but also the surface of any accessible porous regions within the solid phase material.

In another embodiment the materials further comprise a non-covalently associated nucleic acid binding portion at or near the surface which permits capture and binding of nucleic acid molecules of varying lengths. The non-covalently associated nucleic acid binding portion is associated with the solid matrix by electrostatic attraction to an oppositely charged residue on the surface or is associated by hydrophobic attraction with the surface.

The matrix material of these materials carrying covalently or non-covalently attached nucleic acid binding groups can be any suitable substance. Preferred matrix materials are selected from silica, glass, insoluble synthetic polymers, insoluble polysaccharides, and metallic materials selected from metals, metal oxides, and metal sulfides as well as magnetically responsive materials coated with silica, glass, synthetic polymers, or insoluble polysaccharides. Exemplary materials include silica based materials coated or functionalized with covalently attached surface functional groups that serve to disrupt cells and attract nucleic acids. Also included are suitably surface-functionalized carbohydrate based materials, and polymeric materials having this surface functionality. The surface functional groups serving as nucleic acid binding groups include any groups capable of disrupting cells' structural integrity, and causing attraction of nucleic acid to the solid support. Such groups include, without limitation, hydroxyl, silanol, carboxyl, amino, ammonium, quaternary ammonium and phosphonium salts and ternary sulfonium salt type materials described below. Solid phase materials incorporating amino groups which are protonated at a first lower pH for binding and deprotonated at a second higher pH during release of bound nucleic acid, e.g. materials disclosed in European Patent Specification EP01036082B1, are considered to be within the scope of the solid phase materials useful in the present invention.

For many applications it is preferred that the solid phase material be in the form of particles. Preferably the particles are of a size less than about 50 μm and more preferably less than about 10 μm. Small particles are more readily dispersed in solution and have higher surface/volume ratios. Larger particles and beads can also be useful in methods where gravitational settling or centrifugation are employed.

The solid phase preferably can further comprise a magnetically responsive portion which will usually be in the form of paramagnetic or superparamagnetic microparticles that can be attracted and manipulated by a magnetic field. Such magnetic microparticles comprise a magnetic metal oxide or metal sulfide core, which is generally surrounded by an adsorptively or covalently bound layer to shield the magnetic component. Nucleic acid binding groups can be covalently bound to this layer thereby coating the surface. The magnetic metal oxide core is preferably iron oxide or iron sulfide, wherein iron is Fe²⁺ or Fe³⁺ or both. Magnetic particles enclosed within an organic polymeric layer are disclosed, e.g., in U.S. Pat. Nos. 4,654,267, 5,411,730, and 5,091,206 and in a publication (Tetrahedron Lett., 40 (1999), 8137-8140).

Commercially available magnetic silica or magnetic polymeric particles can be used as the starting materials in preparing magnetic solid phase binding materials useful in the present invention. Suitable types of polymeric particles having surface carboxyl groups are known by the trade names SeraMag™ (Seradyn) and BioMag™ (Polysciences and Bangs Laboratories). A suitable type of silica magnetic particles is known by the trade name MagneSil™ (Promega). Silica magnetic particles having carboxy or amino groups at the surface are available from Chemicell GmbH (Berlin).

Applicant has prepared magnetically responsive particulate binding materials in accordance with the present invention by linking bare or coated metallic cores with an organic linker group to which is linked a nucleic acid binding (NAB) portion. When using a coated metallic core, a convenient coated core is a silica-coated magnetic core or a glass-coated magnetic core. A preferred magnetically responsive metallic core is provided by magnetite, Fe₃O₄. Magnetite can be acquired commercially or prepared by reaction of iron (II) and iron (III) salts in basic solution according to generally known methods.

Linker groups containing at one terminus a trialkoxysilane group can be attached to the surface of metallic materials or coated metallic materials such as silica or glass-coated magnetic particles. Preferred trialkoxysilane compounds have the formula R¹—Si(OR)₃, wherein R is lower alkyl and R¹ is an organic group selected from straight chains, branched chains and rings and comprises from 1 to 100 atoms. The atoms are preferably selected from C, H, B, N, O, S, Si, P, halogens and alkali metals. Representative R¹ groups are 3-aminopropyl, 2-cyanoethyl and 2-carboxyethyl, as well as groups containing cleavable moieties as described more fully below. In a preferred embodiment, a trialkoxysilane compound comprises a cleavable central portion and a reactive group terminal portion, wherein the reactive group can be converted in one step to a quaternary or ternary onium salt by reaction with a tertiary amine, a tertiary phosphine or an organic sulfide. Such linker groups can be installed on the surface of metallic particles and glass or silica-coated metallic particles in a process using fluoride ion. The reaction can be performed in organic solvents including the lower alcohols and aromatic solvents including toluene. Suitable fluoride sources have appreciable solubility in such organic solvents and include cesium fluoride and tetraalkylammonium fluoride salts.

The NAB groups contained in some of the solid phase binding materials useful in the methods of the present invention may serve dual purposes. NAB groups attract and bind nucleic acids, polynucleotides and oligonucleotides of various lengths and base compositions or sequences. They may also serve in some capacity to free nucleic acid from the cellular envelope. Nucleic acid binding groups include, for example, carboxyl, amine and ternary or quaternary onium groups or mixtures of more than one of these groups. Amine groups can be NH₂, alkylamine, and dialkylamine groups. Preferred NAB groups are ternary or quaternary onium groups including quaternary trialkylammonium groups (-QR₃⁺), phosphonium groups (-QR₃⁺) including trialkylphosphonium or triarylphosphonium or mixed alkyl aryl phosphonium groups, and ternary sulfonium groups (-QR₂⁺). The solid phase can contain more than one kind of nucleic acid binding group as described herein. Solid phase materials containing ternary or quaternary onium groups-QR₂⁺ or -QR₃⁺ wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Most known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

Some embodiments employ solid phase binding materials in which the NAB groups are attached to the matrix through a linkage which can be selectively broken. Breaking the link effectively “disconnects” any bound nucleic acids from the solid phase. The link can be cleaved by any chemical, enzymatic, photochemical or other means that specifically breaks bond(s) in the cleavable linker but does not also destroy the nucleic acids of interest. Such cleavable solid phase materials comprise a solid support portion comprising a matrix selected from silica, glass, insoluble synthetic polymers, insoluble polysaccharides, and metallic materials selected from metals, metal oxides, and metal sulfides. A nucleic acid binding (NAB) portion for attracting and binding nucleic acids is attached to a surface of the solid support by a cleavable linker portion.

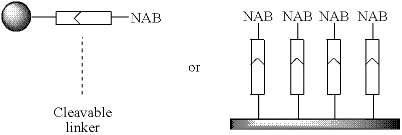

In a preferred embodiment the NAB is a ternary onium group of the formula QR₂⁺ X⁻ or a quaternary onium group QR₃⁺ X⁻ as described above.

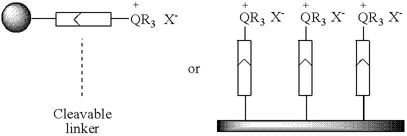

The cleavable linker portion is preferably an organic group selected from straight chains, branched chains and rings and comprises from 1 to 100 atoms. The atoms are preferably selected from C, H, B, N, O, S, Si, P, halogens and alkali metals. An exemplary linker group is a hydrolytically cleavable group which is cleaved by hydrolysis. Carboxylic esters and anhydrides, thioesters, carbonate esters, thiocarbonate esters, urethanes, imides, sulfonamides, and sulfonimides are representative as are sulfonate esters. In a preferred embodiment the cleavable link is treated with an aqueous alkaline solution. Another exemplary class of linker groups are those groups which undergo reductive cleavage such as a disulfide (S—S) bond which is cleaved by thiols such as ethanethiol, mercaptoethanol, and DTT. Another representative group is an organic group containing a peroxide (O—O) bond. Peroxide bonds can be cleaved by thiols, amines and phosphines.

Another representative group is a photochemically cleavable linker group such as nitro-substituted aromatic ethers and esters of the formula 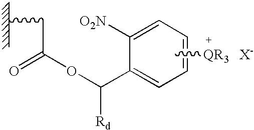
where R_{d} is H, alkyl or phenyl. Ortho-nitrobenzyl esters are cleaved by ultraviolet light according to the well known reaction below.

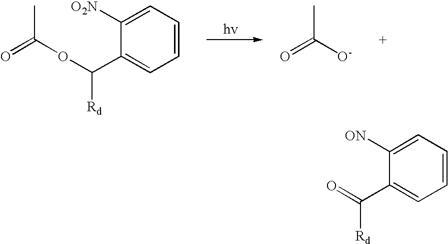

Another representative cleavable group is an enzymatically cleavable linker group. Exemplary groups include esters which are cleaved by esterases and hydrolases, amides and peptides which are cleaved by proteases and peptidases, glycoside groups which are cleaved by glycosidases.

Another representative cleavable group is a cleavable 1,2-dioxetane moiety. Such materials contain a dioxetane moiety which can be decomposed thermally or triggered to fragment by a chemical or enzymatic reagent. Removal of a protecting group to generate an oxyanion promotes decomposition of the dioxetane ring. Fragmentation occurs by cleavage of the peroxidic O—O bond as well as the C—C bond according to a well known process. Cleavable dioxetanes are described in numerous patents and publications. Representative examples include U.S. Pat. Nos. 4,952,707, 5,707,559, 5,578,253, 6,036,892, 6,228,653 and 6,461,876.

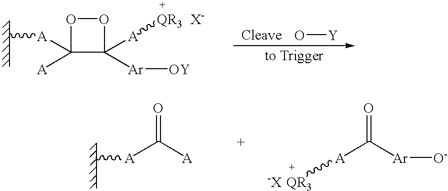

In the alternative, the linked onium group can be attached to the aryl group Ar or to the cleavable group Y. In a further alternative, the linkages to the solid phase and ternary or quaternary onium groups are reversed from the orientation shown.

Another cleavable linker group is an electron-rich C—C double bond which can be converted to an unstable 1,2-dioxetane moiety. At least one of the substituents on the double bond is attached to the double bond by means of an O, S, or N atom. Reaction of electron-rich double bonds with singlet oxygen produces an unstable 1,2-dioxetane ring group which spontaneously fragments at ambient temperatures to generate two carbonyl fragments. Unstable dioxetanes formed from electron-rich double bonds are described in numerous patents and publications exemplified by A. P. Schaap and S. D. Gagnon, J. Am. Chem. Soc., 104, 3504-6 (1982); W. Adam, Chem. Ber., 116, 839-46, (1983); U.S. Pat. No. 5,780,646.

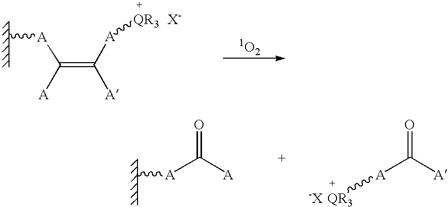

Another group of solid phase materials having a cleavable linker group have as the cleavable moiety a ketene dithioacetal as disclosed in U.S. Pat. Nos. 6,858,733 and 6,872,828. Ketene dithioacetals undergo oxidative cleavage of a double bond by enzymatic oxidation with a peroxidase enzyme and hydrogen peroxide. 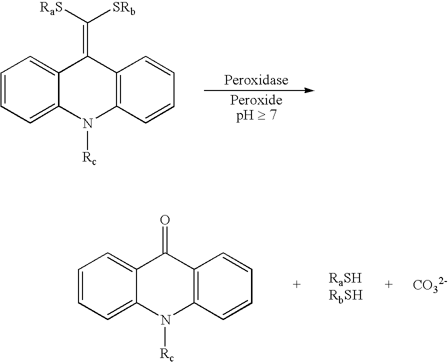
The cleavable moiety has the structure shown, including analogs having substitution on the acridan ring, wherein Rₐ R_{b} and R_{c} are each organic groups containing from 1 to about 50 non-hydrogen atoms selected from C, N, O, S, P, Si and halogen atoms and wherein Rₐ and R_{b} can be joined together to form a ring. Numerous other cleavable groups will be apparent to the skilled artisan. To use this type of compound as a cleavable linker, R_{c} may be attached to the solid phase while Rₐ and/or R_{b} is attached to the NAB group or vice versa. Such compounds are also cleavable by a simple and rapid chemical oxidative process described in U.S. Pat. No. 6,126,870.

In a preferred embodiment the solid phase comprises a matrix selected from silica, glass, insoluble synthetic polymers, insoluble polysaccharides, and magnetically responsive versions of these matrices and an onium group attached on a surface of the matrix selected from a ternary sulfonium group of the formula QR₂⁺ X⁻ where R is selected from C₁-C₂₀ alkyl, aralkyl and aryl groups, a quaternary ammonium group of the formula NR₃⁺ X⁻ wherein R is selected from C₁-C₂₀ alkyl, aralkyl and aryl groups, and a quaternary phosphonium group PR₃⁺ X⁻ wherein R is selected from C₁-C₂₀ alkyl, aralkyl and aryl groups, and wherein X is an anion,

4. Elution Reagents of the Invention

According to the methods of the invention RNA is eluted from the solid phase by contacting the solid phase material with a reagent to release the bound RNA into solution. The solution should dissolve and sufficiently preserve the released RNA. In one embodiment the solution can be a reagent composition comprising an aqueous buffer solution having a pH of about 7-9, optionally containing 0.1-3 M, buffer salt, metal halide or acetate salt and optionally containing an organic co-solvent at 0.1-50% or a surfactant.

In another embodiment the reagent for releasing the nucleic acid from the solid phase binding material does so after cleavage of a cleavable linker group present in the solid phase binding material. A preferred reagent is a strongly alkaline aqueous solution. Solutions of alkali metal hydroxides or ammonium hydroxide at a concentration of at least 10⁻⁴ M are effective in cleaving and eluting nucleic acid from the cleaved solid phase. Strongly alkaline solutions are useful in conjunction with solid phase binding materials in which the nucleic acid binding portion is attached to the matrix through a group which can be fragmented or cleaved by covalent bond breakage. Such materials are described above and in the aforementioned co-pending U.S. patent application Ser. Nos. 10/714,763, 10/715,284, 10/891,880, and 11/061,984. An exemplary material comprises a cleavable ester or thioester linking group linked to a nucleic acid-attracting onium group such as a quaternary phosphonium group.

The release step can be performed at room temperature, but any convenient temperature can be used. Elution temperature does not appear to be critical to the success of the present methods of isolating nucleic acids. Ambient temperature is preferred, but elevated temperatures may increase the rate of elution in some cases.

5. Kits of the Invention

One or more of the above-described Agent solutions may be provided in a kit along with the solid phase material and elution reagent for isolating ribonucleic acid from a biological sample. Optionally the reagents for accomplishing the additional purification steps identified above may also be included in such a kit for ready performance of this method. Other conventional components of kits for such isolation methods may also be included in a kit.

A kit for isolating RNA from a sample in accordance with the invention can contain, for example, an Agent of the formula: 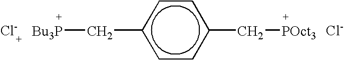
as the free substance or in a solution such as an aqueous solution, a solid phase binding material such as the phosphonium-substituted material described in Example 2, an elution reagent comprising 50 mM NaOH, and an optional wash buffer such as 10 mM Tris, pH 8.0.

### EXAMPLES

### Example 1

### Synthesis of 4′-Hydroxyphenyl 4-chloromethyl-thiobenzoate

A 3 L flask was charged with 100.9 g of 4-chloromethyl-benzoic acid and 1.2 L of thionyl chloride. The reaction was refluxed for 4 h, after which the thionyl chloride was removed under reduced pressure. Residual thionyl chloride was removed by addition of CH₂Cl₂ and evaporation under reduced pressure.

A 3 L flask containing 113.1 g of 4-chloromethylbenzoic acid chloride was charged with 98.17 g of 4-hydroxy-thiophenol and 1.5 L of CH₂Cl₂. Argon was purged in and 67.75 mL of pyridine added. After stirring over night, the reaction mixture was diluted with 1 L of CH₂Cl₂ and extracted with a total of 5 L of water. The water layer was back extracted with CH₂Cl₂. The combined CH₂Cl₂ solutions were dried over sodium sulfate and concentrated to a solid. The solid was washed with 500 mL of CH₂Cl₂ filtered and air dried. ¹H NMR (acetone-d₆): δ 4.809 (s, 2H), 6.946-6.968 (d, 2H), 7.323-7.346 (d, 2H), 7.643-7.664 (d, 2H), 8.004-8.025 (d, 2H).

### Example 2

### Synthesis of Magnetic Silica Particles Functionalized with Polymethacrylate Linker and Containing Tributylphosphonium Groups and Cleavable Arylthioester Linkage

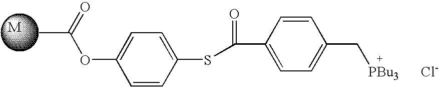

Magnetic carboxylic acid-functionalized silica particles (Chemicell, SiMAG-TCL, 1.0 meq/g, 1.5 g) were placed in 20 mL of thionyl chloride and refluxed for 4 hours. The excess thionyl chloride was removed under reduced pressure. The resin was resuspended in 25 mL of CHCl₃ and the suspension dispersed by ultrasound. The solvent was evaporated and ultrasonic wash treatment repeated. The particles were dried under vacuum for further use.

The acid chloride functionalized particles were suspended in 38 mL of CH₂Cl₂ along with 388 mg of diisopropylethylamine. 4′-Hydroxyphenyl 4-chloromethyl-thiobenzoate (524 mg) was added and the sealed reaction flask left on the shaker over night. The particles were transferred to a 50 mL plastic tube and washed repeatedly, with magnetic separation, with portions of CH₂Cl₂/CH₃OH, 1:1 CH₂Cl₂/CH₃OH, and then CH₂Cl₂. Wash solutions were monitored by TLC for removal of unreacted soluble starting materials. The solid was air dried before further use.

The resin (1.233 g) was suspended in 20 mL of CH₂Cl₂ under argon. Tributylphosphine (395 mg) was added and the slurry shaken for 7 days. The particles were transferred to a 50 mL plastic tube and washed 4 times with 40 mL of CH₂Cl₂ followed with 4 washes of 40 mL of MeOH and 4 times with 40 mL of CH₂Cl₂. The resin was then air dried yielding 1.17 g of a light brown solid.

### Example 3

### Agent Compounds Used in Isolation of Nucleic Acids

A compound having the formula: 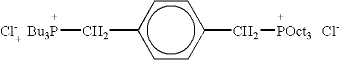
and designated Compound 1 was evaluated for its effect on nucleic acid binding and release. The preparation of Compound 1 was described in U.S. Pat. No. 5,439,617.

Compounds 2 and 3 are polymers prepared as described in U.S. Pat. No. 5,393,469. 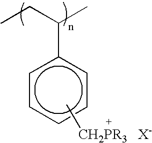
Compound 2: R=n-butyl; Compound 3: R=3:1 ratio of n-butyl and n-octyl; X is Cl; substitution is predominantly in the para-position.

### Example 4

### Recovery of Various Nucleic Acids According to the Invention

Two μg quantities of either E. coli genomic DNA, pUC18 plasmid DNA, or Luciferase RNA were dissolved in 200 μL of deionized water (control) or a 1 mg/mL (0.1%) solution of Compound 1. A 200 μL aliquot of each solution was added to 1 mg of particles of Example 2 and the mixture vortex mixed for 30 s. The solution was removed and the particles were washed with 2×200 μL of 10 mM Tris, pH 8.0. Nucleic acid was eluted from the particles with 1×100 μL 0.1 M NaOH solution at 37° C. by rocking the tubes for 5 min. Eluents were run on a 1% agarose gel with ethidium bromide staining.

Deionized water, if not treated by a process which guarantees the absence of biological activity from RNase should be treated to inactivate RNase activity e.g., by treatment with diethylpyrocarbonate (DEPC).

Gel Results

| Test solution | gDNA | Plasmid | RNA |
|---|---|---|---|
| Compound 1 | − | +/− | + |
| Control | + | + | + |

− not detected;
+/− very slight;
+ positive;
++ strong;
+++ very intense band

Addition of Compound 1 to the binding solutions prevented or reduced the isolation of genomic or plasmid DNA.

### Example 5

### General Protocol for Nucleic Acid Isolation from Bacterial Culture

DH5α (E. coli genomic DNA only) and E. coli/pUC18, JM109 strains (E. coli genomic DNA and pUC18 plasmid DNA) were used in the following experiments.

A 1 mL aliquot of culture (thoroughly mixed) was spun down at 4° C., 7000 rpm for 10 min. Medium was discarded from all pellets. The pellet was resuspended in 1 mL of deionized water. A 200 μL aliquot of the suspension was added to 1 mg of beads of Example 2 and the mixture vortex mixed for 30 s. The solution was removed and the beads were washed with 2×200 μL of 10 mM Tris, pH 8.0. Nucleic acid was eluted from the beads with 1×100 μL 0.1 M NaOH solution at 37° C. by rocking the tubes for 5 min. Eluents were run on a 1% agarose gel with ethidium bromide staining and were analyzed by fluorescence using PicoGreen™.

### Example 6

### Effect of Salt in Resuspension/Binding Solution on Isolation of Nucleic Acid

Using both DH5μ and E. coli/pUC18 culture, resuspension of the pellet was tested in deionized water (control) and titrated concentrations of NaCl. The concentrations of NaCl included 1, 0.5, 0.25, and 0.1 M NaCl. Gel and fluorescence results indicated that the higher the NaCl concentration, the lower the binding and lower the elution. Genomic DNA and plasmid bands were observed on the gel. The test conditions showed no selectivity for isolating RNA.

### Example 7

### Effect of Nonionic Surfactant in Resuspension/Binding Solution on Isolation of Nucleic Acid

Using both DH5μ and E. coli/pUC18 culture, resuspension of the pellet was tested in deionized water (control) and titrated percentages of Tween-20. The percentages of Tween-20 included 5, 1, 0.1, and 0.01%. Gel and fluorescence results indicated that the higher the percentage of Tween-20, the more nucleic acid was removed during the wash steps and the lower the elution. Genomic DNA, plasmid, and RNA bands were visible on the gel. The test conditions showed no selectivity for isolating RNA.

### Example 8

### Effect of Agent in Resuspension/Binding Solution on Isolation of Nucleic Acid from DH5α Culture

Compound 1 was evaluated by the general protocol for its effect on nucleic acid binding and release. Pelleted DH5α culture was resuspended in deionized water (control) or various concentrations of Compound 1 prepared in deionized water. The concentrations of Compound 1 included 1, 0.5, 0.1, and 0.01%. Fluorescence correlated with gel, however, very intense RNA is saturating the fluorescence readings.

Gel Results

| [Compound 1] | gDNA | RNA |
|---|---|---|
| 1% | − | − |
| 0.5% | − | +++ |
| 0.1% | − | +++ |
| 0.01% | − | + |
| 0 (water) | + | ++ |

### Example 9

### Effect of Agent in Resuspension/Binding Solution on Isolation of Nucleic Acid from E. coli/pUC18 Culture

The protocol of Example 8 was followed using a culture of E. coli/pUC18.

Gel Results

| [Compound 1] | gDNA | Plasmid | RNA |
|---|---|---|---|
| 1% | − | − | − |
| 0.5% | − | − | +++ |
| 0.1% | +/− | +/− | +++ |
| 0.01% | + | + | + |
| 0 (water) | + | + | ++ |

### Example 10

### Effect of RNase a Treatment

The isolation procedures of Examples 8 and 9 were repeated, resuspending the pellets in either deionized water (control) or 1 mg/mL of Compound 1. An aliquot of each eluent was immediately treated with RNase A (final concentration in eluent 0.01%). Controls and RNase-treated eluents were analyzed after gel electrophoresis. Samples treated with RNase A exhibited an RNA band on the gel diminished at least 10-fold, confirming the identity of the bands as RNA.

### Example 11

### Effect of Different Agents in Resuspension/Binding Solution on Isolation of Nucleic Acid from DH5α Culture

Additional Agents were tested in the resuspension/binding solutions. All resuspension buffers were prepared in deionized water containing 0.1% DEPC. Following the protocol above with DH5α culture, pellets were resuspended in either deionized water (control), 0.1% Compound 1, 0.1% Compound 2, 0.1% Compound 3, 0.1% CTAB, or 0.1% tetrabutylammonium bromide (Bu₄NBr).

According to fluorescence values, binding in Compound 2, Compound 3 and CTAB was lower than the remaining resuspension buffers, and therefore the lower the elution counts.

Gel Results

| Agent | gDNA | RNA |
|---|---|---|
| Compound 1 | − | +++ |
| Compound 2 | − | + |
| Compound 3 | − | ++ |
| CTAB | − | − |
| Bu₄NBr | + | + |
| Control | + | + |

### Example 12

### Effect of Different Agents in Resuspension/Binding Solution on Isolation of Nucleic Acid from E. coli/pUC18 Culture

The procedure of Example 11 was performed on an E. coli/pUC18 culture.

Gel Results

| Agent | gDNA | Plasmid | RNA |
|---|---|---|---|
| Compound 1 | +/− | +/− | +++ |
| Compound 2 | − | − | + |
| Compound 2 | − | − | + |
| CTAB | − | − | − |
| Bu₄NBr | + | + | ++ |
| Control | + | + | ++ |

### Example 13

### Effect of Different Agent Concentrations in Resuspension/Binding Solution on Isolation of Nucleic Acid from DH5α Culture

A further investigation of the range of useful concentrations of Compounds 1 and 2 in the present methods was made as described above. Results were compared to a similar study using CTAB.

| [Compound 1] | gDNA | RNA |
|---|---|---|
| 0.50% | − | +++ |
| 0.375% | − | +++ |
| 0.25% | − | +++ |
| 0.10% | − | +++ |
| 0.05% | − | +++ |
| 0 (water) | + | ++ |

| [Compound 2] | gDNA | RNA |
|---|---|---|
| 0.1% | − | + |
| 0.05% | − | + |
| 0.01% | − | ++ |
| 0.05% | − | ++ |
| 0.001% | + | ++ |
| 0 (water) | + | ++ |

| [CTAB] | gDNA | RNA |
|---|---|---|
| 0.1% | − | − |
| 0.05% | + | + |
| 0.01% | + | ++ |
| 0.005% | + | ++ |
| 0.001% | + | ++ |
| 0 (water) | + | ++ |

### Example 14

### Effect of Different Agent Concentrations in Resuspension/Binding Solution on Isolation of Nucleic Acid from E. coli/pUC18 Culture

A further investigation of the range of useful concentrations of Compounds 1 and 2 in the present methods was made as described above. Results were compared to a similar study using CTAB. Use of CTAB did not enhance recovery of RNA.

| [Compound 1] | gDNA | Plasmid | RNA |
|---|---|---|---|
| 0.50% | − | − | +++ |
| 0.375% | − | − | +++ |
| 0.25% | − | +/− | +++ |
| 0.10% | +/− | +/− | +++ |
| 0.05% | + | + | +++ |
| 0 (water) | ++ | ++ | ++ |

| [Compound 2] | gDNA | Plasmid | RNA |
|---|---|---|---|
| 0.1% | − | − | + |
| 0.05% | − | − | ++ |
| 0.01% | − | − | ++ |
| 0.05% | +/− | +/− | ++ |
| 0.001% | ++ | ++ | ++ |
| 0 (water) | ++ | ++ | ++ |

| [CTAB] | gDNA | Plasmid | RNA |
|---|---|---|---|
| 0.1% | − | − | − |
| 0.05% | + | − | + |
| 0.01% | + | + | ++ |
| 0.005% | + | + | ++ |
| 0.001% | ++ | ++ | ++ |
| 0 (water) | ++ | ++ | ++ |

### Example 15

### Testing of Culture Pellet Size

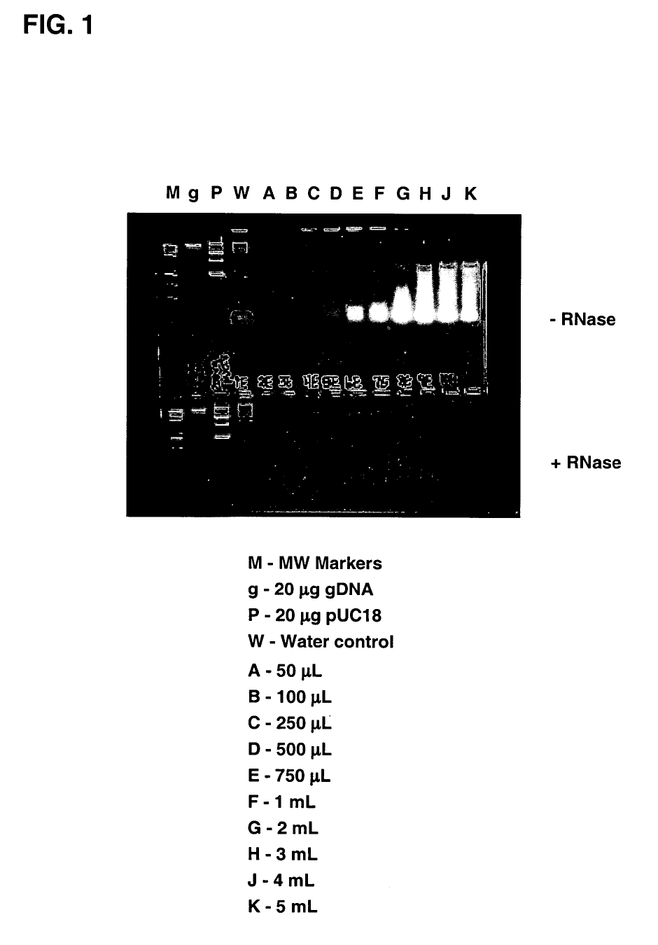

Various volumes of culture were processed according to the general protocol in order to further evaluate the effectiveness of the method. The resuspension/binding solution contained Compound 1 at a concentration of 0.5% in deionized water. Culture aliquots processed were 1 mL of E. Coli/pUC18 culture resuspended in 1 mL of deionized water (control) and 50 μL, 100 μL, 250 μL, 500 μL, 750 μL, 1 mL, 2 mL, 3 mL, 4 mL, and 5 mL of E. Coli/pUC18 culture pelleted and resuspended in 1 mL of 0.5% Compound 1. The gel results shown in FIG. 1 reveal that RNA was isolated selectively in as little as 250 μL of culture under these conditions.

Treating an aliquot of each eluent with RNase A (0.02%) confirmed the identity of the band as RNA.

### Example 16

### RNA Isolation from Listeria innocua

The protocol as described in Example 5 in conjunction with E. coli culture was applied to cultures of L. innocua. All L. innocua tests employed 1 mg of the beads of Example 2; pellets from 1 mL of culture were treated with water controls using DEPC treated deionized water or with solutions of Compound 1.

Additionally 2 μg samples of purified L. innocua genomic DNA were bound in either water or 0.5% Compound 1 with either 30 s vortex binding or 5 min room temperature binding.

| From culture | gDNA | RNA |
|---|---|---|
| water control | + | + |
| 0.5% Compound 2 | − | − |

| From Purified L. innocua | gDNA | RNA |
|---|---|---|
| water control, 30 s vortexed | + | NA |
| 0.5% Compound 1, 30 s vortexed | − | NA |
| water control/5′ RT | + | NA |
| 0.5% Compound 1/5′ RT | − | NA |

### Example 17

### Different Agent Concentrations in Resuspension/Binding Solution on Isolation of Nucleic Acid from L. innocua Culture

Resuspension/binding of L. innocua was tested with solutions of Compound 1 ranging from 0.005-0.5%.

| [Compound 1] | gDNA | RNA |
|---|---|---|
| 0.5% | − | − |
| 0.1% | − | − |
| 0.05% | − | + |
| 0.01% | − | ++ |
| 0.005% | +/− | ++ |
| 0 (water) | + | + |

### Example 18

### Testing of Culture Pellet Size

Resuspension/binding of L. innocua was tested with solutions of Compound 1 ranging from 0.005-0.5% and pellets from 1 and 5 mL of culture.

| 1 mL culture pellet | gDNA | RNA |
|---|---|---|
| 0.5% Compound 1 | − | − |
| 0.1% | − | + |
| 0.05% | − | + |
| 0.01% | − | + |
| 0.005% | − | ++ |
| 0 (water) | + | + |

| 5 mL culture pellet | gDNA | RNA |
|---|---|---|
| 0.5% Compound 1 | − | + |
| 0.1% | − | ++ |
| 0.05% | − | ++ |
| 0.01% | − | ++ |
| 0.005% | − | ++ |
| 0 (water) | ++ | ++ |

### Example 19

### Compatibility with Guanidinium Salts

The protocol of Example 5 was repeated with a modification of the pellet resuspension step incorporating guanidinium hydrochloride and heating. Specifically, pellets from 1 mL of E. coli/pUC18 culture were taken up in 0.33 mL of 0.5% Compound 1, and 0.33 mL of 0.7 M guanidinium HCl, heated at 70° C. for 10 min, and treated with 0.33 mL of either ethanol or deionized water prior to binding on the particles. As shown in the table below, the presence of guanidinium in the resuspension/binding solution did not interfere with isolation of RNA.

Gel Results

| | gDNA | Plasmid | RNA |
|---|---|---|---|
| EtOH | − | − | +++ |
| H₂O | − | − | +++ |

### Example 20

### Synthesis of Magnetic Silica Particles Functionalized with Polymethacrylate Linker and Containing Tributylphosphonium Groups and Cleavable Arylthioester Linkage

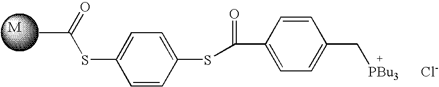

The acid chloride functionalized particles of example 2, prepared from 0.75 g of the COOH-functionalized starting material, were suspended in 40 mL of CH₂Cl₂ along with 0.15 g of 1,4-benzenedithiol and 0.40 mL of diisopropylethyl-amine. The mixture was sonicated for 5 min and agitated with an orbital shaker over night. The solids were washed several times, using magnetic separation, with CH₂Cl₂, 1:1 CH₂Cl₂/CH₃OH, and CH₃OH.

A mixture of the particles of the preceding step (ca. 0.7 g) and 25 mL of CH₂Cl₂ was treated with 0.19 g of 4-chloromethylbenzoyl chloride and 0.40 mL of diisopropyl-ethylamine. The mixture was sonicated for 5 min and agitated with an orbital shaker over night. The solids were washed several times, using magnetic separation, with CH₂Cl₂, 1:1 CH₂Cl₂/CH₃OH, and CH₃OH. Solid collected was dried over night.

A mixture of the particles of the preceding step (ca. 0.63 g) and 25 mL of CH₂Cl₂ was treated with 0.30 g of tributylphosphine. The mixture was sonicated for 5 min and agitated with an orbital shaker for a total of 10 days. The solids were washed several times, using magnetic separation, with CH₂Cl₂, 1:1 CH₂Cl₂/CH₃OH, and CH₃OH. Solid collected was dried over night.

### Example 21

### Isolation of RNA from Human Blood

The following protocol was used to isolate RNA from whole blood samples (drawn into Acid Citrate Dextrose tubes).

Ten samples (100 μL) of freshly drawn whole blood was added to 60 μg Proteinase K and 100 μL 0.7M guanidine HCl/20% Tween-20. Each solution was rocked for 10 min at room temperature. Each sample tube was treated with 100 μL of a 0.5% solution of Compound 1 prepared in DEPC treated deionized water. A 200 μL portion of the resulting solutions were each applied to 2 mg of the particles of example 20. Nucleic acid was bound for 5 min at room temperature. Particles were washed with 3×500 μL of 0.7M guanidine HCl/10% Tween-20/50% EtOH washes, 2×200 μL of DEPC treated deionized water. RNA was eluted with 50 μL of 50 mM NaOH solution for 5 min at room temperature.

Eluents from this first step showed both genomic DNA and RNA bands present on the agarose gel.

Five of the eluents were run through a second binding and elution protocol by diluting 50 μL of the eluents with 50 μL of 0.3 M KOAc, 0.2 M HCl, 0.05% Compound 1. The solutions were bound to 2 mg of the particles of example 20 for 5 min at room temperature. Particles were washed and eluted as above. After the second binding/elution gel analysis revealed only the RNA band in four of the five samples.

### Example 22

### Isolation of RNA from Culture

The following two step protocol was used to isolate RNA from DH5α and E. coli/pUC18.

Duplicate one mL samples of thoroughly mixed culture were spun down at 7000 rpm for 10 min at 4° C. Media was discarded from the pellets and the pellets were resuspended in 1 mL of either water or a solution of 0.5% Compound 1. RNA was bound by adding 200 μL of pellet suspensions to 2 mg of the particles of example 20 and vortex mixing for 30 s. The particles were washed with 2×200 μL of 10 mM tris, pH 8.0 and eluted with 100 μL of 0.1 M NaOH for 5 min at 37° C.

One of the duplicates from each sample was run through a second binding and elution protocol by diluting 100 μL of the eluents with 100 μL of S3+ solution: 0.3 M KOAc, 0.2 M HCl, 0.05% Compound 1. The solutions were bound to 2 mg of the particles of example 20 for 30 s at room temperature. Particles were washed and eluted as above. After the second binding/elution gel analysis revealed only the RNA band in four of the five samples.

| DH5α | gDNA | Plasmid | RNA |
|---|---|---|---|
| water (1st elute) | +++ | NA | +++ |
| water/S3+ (2nd elute) | + | NA | ++ |
| 0.05% Compound 1 (1st elute) | − | NA | ++ |
| S3+ (2nd elute) | − | NA | + |

E. coli/pUC18

| | | | |
|---|---|---|---|
| water (1st elute) | +++ | +++ | +++ |
| water/S3+ (2nd elute) | + | + | +++ |
| 0.05% Compound 1 (1st elute) | − | − | +++ |
| S3+ (2nd elute) | − | − | ++ |

Both of examples 21 and 22 demonstrate the ability to enrich or isolate RNA from samples containing mixtures of nucleic acids that have been subjected to previous nucleic acid extraction treatments or conditions.

What is claimed is:

1. A method for isolating RNA from a biological sample comprising the steps of:
(a) forming a mixture of the sample and a solution of an Agent for enhancing recovery of RNA;
(b) contacting the mixture with a solid phase binding material;
(c) separating the solid phase binding material from the solution; and
(d) dissociating the RNA from the solid phase binding material.

2. The method according to claim 1 wherein the biological sample is selected from the group consisting of whole blood, plasma, serum, urine, tissue, cells, bacterial culture and viruses.

3. The method of claim 1 wherein the Agent is multiply charged.

4. The method of claim 3 wherein the multiply charged Agent carries two or more positive charges.

5. The method of claim 4 wherein the Agent has the formula:
X⁻⁺R₃ACH₂-Link-CH₂AR₃⁺X⁻
wherein each A is selected from P and N atoms and wherein Link is an organic spacer group containing two or more carbon atoms selected from the group consisting of substituted and unsubstituted aryl, alkyl, alkenyl, alkynyl and wherein Link may contain heteroatoms and wherein R is selected from lower alkyl or aralkyl containing 1 to 20 carbon atoms and wherein X is an anion.

6. The method of claim 5 wherein the Agent has the formula: 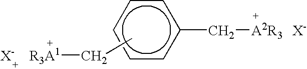
wherein the substituents may be in the ortho-, meta- or para-orientation and wherein A¹ and A¹ are independently chosen from nitrogen or phosphorus atoms, and wherein each R is independently alkyl or aralkyl containing from 1-20 carbon atoms and wherein X is an anion.

7. The method of claim 5 wherein the Agent has the formula:
X⁻⁺R₃A¹CH₂-Link-CH₂A²R₃⁺X⁻
wherein A¹ and A¹ are independently selected from nitrogen or phosphorus atoms and wherein Link contains two or more carbon atoms and is selected from the group consisting of substituted and unsubstituted alkyl, alkenyl, alkynyl, and wherein each R is independently alkyl or aralkyl containing from 1-20 carbon atoms and wherein X is an anion.

8. The method of claim 4 wherein the Agent is a polymer having charged groups as part of the polymer backbone or as pendant groups.

9. The method of claim 8 wherein the polymer has the formula: 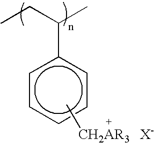
wherein each A is selected from phosphorus and nitrogen atoms, each R is selected from lower alkyl or aralkyl containing 1 to 20 carbon atoms, and wherein n is an integer between about 10 and 1000.

10. The method of claim 9 wherein the polymer has the formula: 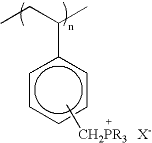
wherein each R is selected from lower alkyl or aralkyl containing 1 to 20 carbon atoms, and wherein n is an integer between about 10 and 1000.

11. The method of claim 1 wherein the binding material is selected from particles, microparticles, fibers, beads, membranes, test tubes and microwells.

12. The method of claim 11 wherein the binding material is in the form of particles of size less than about 50 μm.

13. The method of claim 1 wherein the binding material comprises a matrix material selected from silica, glass, sintered glass, controlled pore glass, sintered glass, alumina, zirconia, titania, insoluble synthetic polymers, insoluble polysaccharides, metals, metal oxides, metal sulfides, and magnetically responsive materials coated with silica, glass, synthetic polymers, or insoluble polysaccharides.

14. The method of claim 13 wherein the binding material further comprise a nucleic acid binding portion covalently linked to the matrix at or near the surface.

15. The method of claim 14 wherein the covalent linkage between the matrix and nucleic acid binding portion is selectively cleaved.

16. The method of claim 14 wherein the nucleic acid binding group is selected from hydroxyl, silanol, carboxyl, amine, ammonium, quaternary ammonium and phosphonium salts and ternary sulfonium salts or mixtures of more than one of these groups.

17. The method of claim 16 wherein the nucleic acid binding group is a quaternary phosphonium group of the formula ^{+PR}₃X⁻ wherein each R is alkyl and X is an anion.

18. The method of claim 14 wherein the covalent linkage between the matrix and nucleic acid binding portion is cleaved by an alkaline eluent to release RNA into the eluent in step d.

19. The method of claim 1 wherein the Agent carries two or more positive charges, wherein the solid phase binding material comprises a nucleic acid binding portion covalently linked to the matrix at or near the surface which is cleaved by an alkaline eluent to release RNA into the eluent in step d.

20. The method of claim 1 wherein the presence of the Agent in step a) allows RNA to be selectively isolated from genomic DNA and plasmid DNA.
